(19) United States
(12) Patent Application Publication
(10) Pub. No.: US 2013/0236568 A1
(43) Pub. Date: Sep. 12, 2013

(54) PHOSPHAPLATINS HAVING ANTI-ANGIOGENIC, ANTI-METASTATIC, AND PRO-APOPTOTIC PROPERTIES AND USES THEREOF

(71) Applicant: Rathindra N. Bose, Houston, TX (US)

(72) Inventor: Rathindra N. Bose, Houston, TX (US)

(73) Assignee: Ohio University, Athens, OH (US)

(21) Appl. No.: 13/989,457

(22) PCT Filed: Dec. 2, 2011
(86) PCT No.: PCT/US11/63139

§ 371 Date: May 24, 2013

Related U.S. Application Data
(60) Provisional application No. 61/431,900, filed on Jan. 12, 2011.

(51) Int. Cl. A61K 31/6615; A61K 45/06; C07F 19/00

(52) U.S. Cl. CPC A61K 31/6615; C07F 19/005; A61K 45/06
USPC 424/649; 514/114; 435/375

(57) ABSTRACT

Provided are compositions and uses thereof in methods of inhibiting angiogenesis, metastasis, or both, wherein said compositions comprise phosphaplatins such as pyrodach-4. In some embodiments, provided are compositions and uses thereof in methods of treating sensitive and resistant cancers.

### CROSS-REFERENCE TO RELATED APPLICATIONS

This application claims priority under 35 U.S.C. §119(e) to U.S. Provisional Patent Application Ser. No. 61/431,900, filed on Jan. 12, 2011, which is hereby incorporated by reference in its entirety.

### FIELD OF THE INVENTION

This application relates to anti-angiogenic, anti-metastatic, and pro-apoptotic properties of phosphaplatins; compositions and uses thereof to inhibit angiogenesis, inhibit metastasis, promote apoptosis, or combinations thereof; and compositions and uses thereof to treat resistant, as well as advanced, cancers.

### BACKGROUND OF THE INVENTION

The value of novel anti-cancer therapies cannot be underestimated, as tens of millions are annually diagnosed with cancer and millions die each year from this malady which represents a predominate cause of death worldwide. Once a cancer is diagnosed, the treatment outcome for the patient depends greatly on factors such as whether the cancer was diagnosed at an early stage, whether the cancer has spread throughout the body, and whether the cancer is or has become resistant to known chemotherapeutic regimens.

The platinum-based anticancer drugs cisplatin, carboplatin, and oxaliplatin, are widely used for treating a variety of cancers such as ovarian cancer, testicular cancer, non-small cell lung cancer, and colorectal cancer. These compounds may be used in combination with other therapeutic regimens, including radiation therapy, to treat an extensive array of cancers. Recent clinical trials in adjuvant therapeutic modes utilizing platinum compounds underscore the potential of platinum compounds to effectively treat a wide variety of other cancers. For example, recent breakthrough research suggests that a diabetic drug, rosiglitazone, may be effectively used in combination with carboplatin to treat multiple forms of cancer. This has now added a new dimension to the ever-growing applications of platinum-based anticancer drugs, because most adjuvant therapies have been limited primarily to combinations of cancer or radiation drugs with other cancer drugs. Thus, there remains an ongoing need for new platinum-based anticancer drugs, as well as new applications for platinum-based anticancer drugs.

Conventional platinum chemotherapeutics, such as cisplatin, initiate apoptosis at the G2 phase of the cell cycle predominantly through transcription inhibition and through replication inhibition processes, especially at high doses. Covalent binding to DNA through the N7 sites of guanine and adenine bases, both by intra-strand and inter-strand modes, is believed to be the key molecular event in triggering a cascade of cellular responses leading to apoptosis (programmed cell death). Numerous challenges have been identified in understanding the complexity of the cellular and molecular metallo-biochemistry of cisplatin and the molecular mechanisms of cytotoxicity. Briefly, it has been noted that platinated DNA is at the heart of the initiation of cytotoxicity. The platinum-bound DNA is sequestered by high mobility proteins (HMG) thus affecting repairs by the nucleotide excision repair (NER) enzymes. Furthermore, these platinum-DNA adducts are believed to activate the p53 transcription factor, to induce histone phosphorylation, and to trigger chromatin condensation.

Although platinum-based chemotherapeutics are widely used to treat cancers, their applications in large numbers of patients have been limited because of severe side effects such as nephrotoxicity, neurotoxicity, ototoxicity, myelosuppression, and acquired resistance to platinum-metal drugs. For example, a significant percentage of patients become resistant to cisplatin treatment. Although carboplatin reduces some toxicity over cisplatin, it does not alleviate the resistance. Currently, oxaliplatin is approved to treat colorectal cancer, but its resistance is largely unexplored.

The art lacks an understanding at the molecular level of the development of resistance to conventional platinum-based chemotherapeutics and ways to overcome such resistance. Although the understanding is incomplete, it is believed that the ability to repair DNA damage by excising bound platinum from DNA mostly contributes to the resistance mechanisms. In light of the aforementioned, there is an ongoing need for ways to overcome resistance to platinum drugs, including development of drugs that are not susceptible to DNA repair mechanisms.

In various embodiments, U.S. Pat. No. 7,700,649 (Bose) and U.S. Ser. No. 12/722,189 (Bose) meet some of the needs in the art by disclosing synthetic routes and cancer treatment methods involving a new class of platinum complexes, namely pyrophosphato complexes having platinum metal centers. The disclosed compounds and methods are part of a drug development strategy based on creating a class of platinum antitumor agents that do not covalently bind DNA, thereby nullifying DNA-repair based resistance. This strategy is a paradigm shift from conventional platinum drug development approaches, in which DNA binding is the central theme in developing more efficient platinum anticancer agents.

Among the pyrophosphato platinum complexes disclosed in U.S. Pat. No. 7,700,649 (Bose) and U.S. Ser. No. 12/722,189 (Bose) are racemic trans-(±)-1,2-cyclohexanediamine(pyrophosphato) platinum(II), which is also referred to as “dach-2” and “pyrodach-2,” and racemic trans-(±)-1,2-cyclohexanediamine-trans-dihydroxo(pyrophosphato) platinum(IV), which is also referred to as “dach-4” and “pyrodach-4.” These racemic complexes were found efficacious for treatments of some cancers. However, much about these compounds remained unknown, including alternative uses thereof. Therefore, while Applicant's prior applications advanced the state of the art, there nevertheless remains an ongoing need for new therapeutic uses of such platinum complexes.

### SUMMARY OF THE INVENTION

The present application meets the ongoing need and further expands the utility of phosphaplatins. In various embodiments, provided are compositions comprising phosphaplatins for use in inhibiting angiogenesis, inhibiting metastasis, promoting apoptosis, or combinations thereof. Additionally provided are methods of using phosphaplatins, and compositions comprising phosphaplatins, in novel approaches to treating cancers wherein treatments can target specific cellular interactions involved in the development and advancement of cancer. For example, the present application provides options for targeting development of new vessels or regrowth or restructuring of existing vessels. In some embodiments, vessel development involving cancer cells may be targeted, as well as vessel growth or restructuring involving endothelial cells providing conditions conducive to tumor development. As another example, the present application provides options for preventing or inhibiting metastasis, thereby leading to better options for localizing and isolating treatments to affected areas.

Various embodiments disclosed herein detail compositions comprising phosphaplatins for use in inhibiting angiogenesis, metastasis, or both. In some embodiments, the provided compositions may comprise:

(a) (1R,2R)-pyrodach-4 having formula (I)

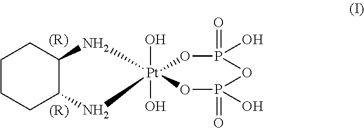

or pharmaceutically acceptable salt or solvate thereof;
(b) (1S,2S)-pyrodach-4 having formula (II)

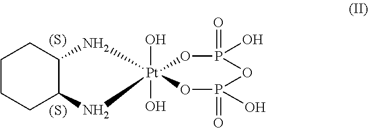

or pharmaceutically acceptable salt or solvate thereof;
(c) cis-pyrodach-4 having formula (III)

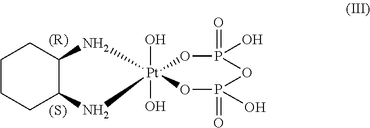

or pharmaceutically acceptable salt or solvate thereof; or
(d) combinations thereof.

In some embodiments, the provided compositions may further comprise at least one pharmaceutically acceptable ingredient selected from carriers, diluents, adjuvants, and vehicles. In some embodiments, the provided compositions may also comprise one or more of cisplatin, carboplatin, and oxaliplatin. In specific embodiments the compositions are administered to induce apoptotic cell death in proliferative cells.

In various embodiments, also provided are contemplated methods of inhibiting angiogenesis, metastasis, or both, using the provided compositions, said methods comprising administering to a subject in need thereof a therapeutically effective amount of a provided composition. In some embodiments, the effective amount administered is sufficient to modify gene expression of at least one cell surface molecule in at least one endothelial cell, cancer cell, or both, of the subject. Examples of suitable cells include, but are not limited to, endothelial or cancer cells of ovarian, brain, stomach, bladder, breast, lung, and pancreatic tissue. In some embodiments, the cancer cells may be resistant to cisplatin, carboplatin, oxaliplatin, or combinations thereof. A non-limiting example of at least one cell surface molecule is E-cadherin, wherein the effective amount administered is administered to increase E-cadherin in at least one cancer cell of the subject.

In some embodiments, the therapeutically effective amount administered of one or more chemicals or compositions described herein is sufficient to decrease gene expression of Vascular Endothelial Growth Factor Receptor-2 (VEGFR-2) surface molecule. In some embodiments, the therapeutically effective amount administered is sufficient to increase gene expression of p53 upregulated modulator of apoptosis (PUMA); phosphatase and tensin-homolog (PTEN); tumor necrosis factor receptor superfamily member 6 (Fas), Fas ligand (FasL); caspase-3 (CASP3); caspase-9 (CASP9); BCL-2-associated X protein (Bax); or combinations thereof. In some of the various embodiments, the effective amount administered is sufficient to increase E-cadherin in at least one cancer cell of the subject. In some embodiments, E-cadherin is increased, while gene expression of Vascular Endothelial Growth Factor Receptor-2 (VEGFR-2) is decreased in at least one cancer cell of the subject.

### BRIEF DESCRIPTION OF THE DRAWINGS

A more complete appreciation of the invention and the many embodiments thereof will be readily obtained as the same becomes better understood by reference to the following detailed description when considered in connection with the accompanying drawings, wherein:

FIG. 1 shows structures for A (R,R)-pyrodach-4; B (S,S)-pyrodach-4; C cis-pyrodach-4; D (R,R)-pyrodach-2; E (S,S)-pyrodach-2; and F cis-pyrodach-2.

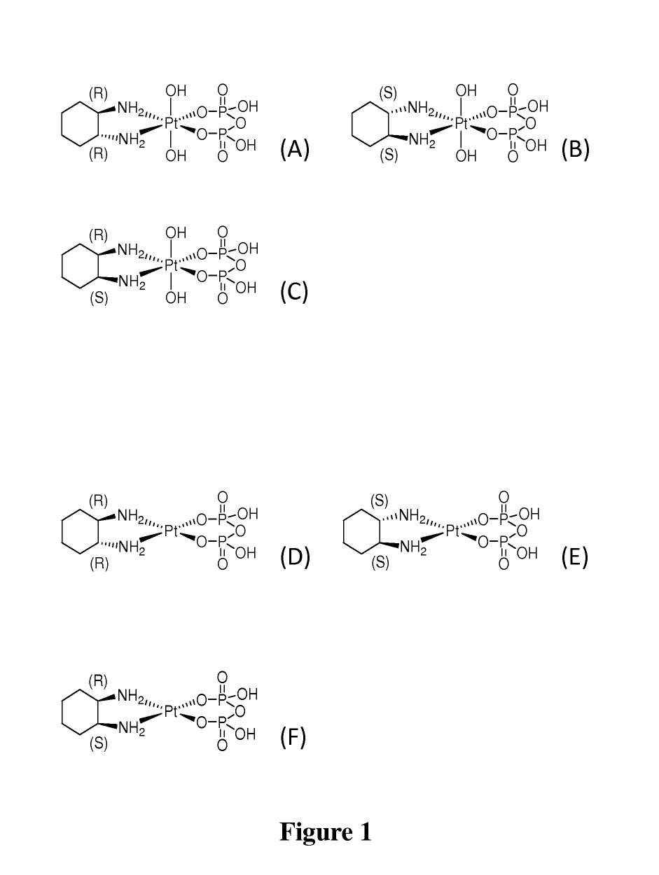

FIG. 2 shows comparative immunofluorescent images of cells stained for the E-cadherin protein (bottom left) before and after treatment of A2780 cells with pyrodach-4 for 24 hours, showing that pyrodach-4 can increase E-cadherin;

FIG. 3 demonstrates steps involved in pooled screens that can be used for identifying gene functions, measuring the functional relatedness of gene pairs in order to group genes into pathways, identifying drug targets, and determining a drug's mechanism of action (such as the effect of pyrodach-4 on strains);

FIG. 4 shows an enzyme-linked immunoabsorbant (ELISA) assay of apoptotic cell death in A2780 cells induced by pyrodach-4 and cisplatin in the absence and presence of p-53 (Pifithrin-alpha (PFT-α), 15 μM) and PI3K (LY294002, 30 μM) inhibitors; and

FIG. 5 shows an enzyme-linked immunoabsorbant (ELISA) of apoptotic cell death in A2780/C30 cells induced by pyrodach-4 and cisplatin in the absence and presence of p-53 (PFT-α, 15 μM) and PI3K (LY294002, 30 μM) inhibitors.

### DESCRIPTION OF EMBODIMENTS

Specific embodiments of the present disclosure will now be described. The invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used herein is for describing particular embodiments only and is not intended to be limiting of the invention. As used in the specification and appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about,” which is intended to mean up to ±10% of an indicated value. Additionally, the disclosure of any ranges in the specification and claims are to be understood as including the range itself and also anything subsumed therein, as well as endpoints. Unless otherwise indicated, the numerical properties set forth in the specification and claims are approximations that may vary depending on the desired properties sought to be obtained in embodiments of the present invention. Notwithstanding that numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

As used herein, the term “phosphaplatin” refers generally to platinum complexes coordinated with a single bidentate pyrophosphato ligand. Phosphaplatins may have the following general structures (A) and (B):

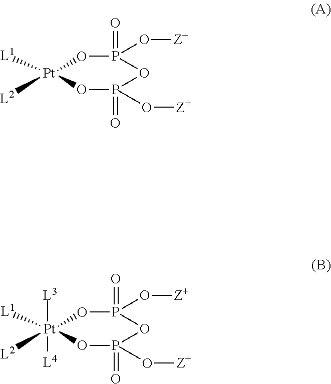

in which L¹ and L² represent neutral ligands (independently selected from NH₃; substituted or unsubstituted aliphatic amines; and substituted or unsubstituted aromatic amines), or a single bidentate neutral ligand (selected from substituted or unsubstituted aliphatic or aromatic diamines) with end groups L¹ and L², coordinated to the platinum metal center; L³ and L⁴ are ligands (selected from hydroxide, acetic acid, butyric acid, and alpha-hydroxy acids, amines or charged species thereof) coordinated to the platinum metal center. The pyrophosphato ligand may be neutral (not shown) or charged (shown). When charged, the pyrophosphato ligand is present with counterions, represented by Z⁺. Examples of Z⁺ include, without limitation, hydrogen; alkali metals such as sodium and potassium; and monovalent organic moieties. Preferably, Z⁺ is a counterion that results in a pharmaceutically acceptable salt. Whether charged or neutral, the general structure of platinum(II) complexes represented by (A) is square-planar, and the general structure of platinum(IV) complexes represented by (B) is octahedral. Non-limiting examples of phosphaplatins represented by (A) are diammine(dihydrogen pyrophosphato)platinum (II); 1,2-ethanediamine(dihydrogen pyrophosphato)platinum (II); and (trans-1,2-cyclohexanediamine)(dihydrogen pyrophosphato)platinum (II). Non-limiting examples of phosphaplatins represented by (B) are cis-diammine-trans-dihydroxo(dihydrogen pyrophosphato)platinum (IV); 1,2-ethanediamine-trans-dihydroxo(dihydrogen pyrophosphato)platinum (IV); and (trans-1,2-cyclohexanediamine)-trans-dihydroxo(dihydrogen pyrophosphato)platinum (IV). In some embodiments, the present application is directed to phosphaplatin complexes represented by (B), wherein the bidentate ligand with end groups L¹ and L² is 1,2-cyclohexanediamine in the cis-configuration or one of the two distinguishable trans-configurations. However, the invention may be embodied in different forms and should not be construed as limited to such embodiments.

In general, phosphaplatins do not readily undergo hydrolysis, are soluble in aqueous solution at neutral pH, and are stable in aqueous solution at neutral pH. Furthermore, phosphaplatins show general cytotoxicity in cancer cell lines, and are effective in cell lines that are resistant to one or both cisplatin and carboplatin. Accordingly, phosphaplatins are effective, and in some cases more effective, in inducing cancer cell death as compared to known platinum cancer drugs, and exhibit desirable stability and solubility in solutions that are suitable for administration to patients. As used herein in reference to the phosphaplatins of the invention, “stable” refers to the resistance of the complexes to hydrolysis when maintained in aqueous solution at a pH in the range from 6-8 for a period of time from between 2 and six days.

Unlike cisplatin, carboplatin, and related platinum-based anti-cancer agents, phosphaplatins do not covalently bind DNA. Resistance to cisplatin, carboplatin, and related platinum anti-cancer agents is believed to originate from the efficient repair of DNA damage by a variety of enzymes including nuclear excision repair enzymes. However, because phosphaplatins do not covalently bind DNA, resistance towards phosphaplatins due to the DNA repair mechanism is unlikely. Data indicates that the cellular binding of phosphaplatins is less than cisplatin, yet phosphaplatins exhibit high cytotoxicity.

Data also indicates that while racemic mixtures of certain phosphaplatins, such as pyrodach-2 and pyrodach-4, are effective for use in treating cancer, enhanced cytotoxicity and greater effectiveness can be achieved with use of complexes that are enantiopure, in an enantiomeric excess, or enantioenriched.

The term “enantiomeric excess” is used herein according to its commonly understood definition. That is, for two enantiomers A and B that may be present in a mixture in molar amounts M_{A} and M_{B}, respectively, the enantiomeric excess E of the enantiomer present in a higher molar amount in the mixture may be expressed by the relation % E=|(M_{A}−M_{B})/(M_{A}+M_{B})×100|, where E>0%. A “racemic mixture” of the enantiomers A and B, which may be designated by abbreviations such as “rac” and/or “(±)” (or simply lack any reference to enantiomers) has E=0% because M_{A}=M_{B}. As a further illustration, a mixture consisting of A and B, in which M_{A}=60% and M_{B}=40%, has an enantiomeric excess of A equal to 20%. The same mixture may be regarded in the alternative as a mixture consisting of 80% racemic mixture of A and B in combination with 20% enantiopure A, inasmuch as each molecule of B (40% of the mixture) may be paired with a molecule of A in the mixture (40% of the mixture) to leave unpaired an excess of molecules of A (20% of the mixture).

As used herein, the term “enantiopure” with regard to a molecule having two enantiomers, A and B, refers to a compound or composition containing substantially only one of the enantiomers A or B, but not both A and B. For an “enantiopure” complex, 97%≦E≦100%.

As used herein, the term “enantioenriched” refers, in, its broadest sense, to a compound or composition containing a molecule having two enantiomers, A and B, such that the compound or composition has an enantiomeric excess of one of the enantiomers, either A or B. Thus, an “enantioenriched mixture of A and B” may refer to a mixture with an enantiomeric excess of A or to a mixture with an enantiomeric excess of B, wherein 0%≦E≦100% for either A or B. As illustrative examples, the enantiomeric excess of either A or B may be greater than 0.01%, greater than 1%, greater than 10%, greater than 25%, greater than 50%, greater than 75%, greater than 90%, greater than 98%, greater than 99%, greater than 99.9%, or even equal to 100%.

In various embodiments, provided herein are stable, monomeric phosphaplatin complexes (and compositions comprising a therapeutically effective amount of one or more of said complexes). In some embodiments, said complexes and compositions may be used in methods of inhibiting angiogenesis. In some embodiments, said complexes and compositions may be used in methods for inhibiting metastasis. In some embodiments, said complexes and compositions may be used in methods of promoting apoptosis. In some embodiments, said complexes and compositions may be used in methods of treating cancers, including but not limited to, cancers resistant to treatment by one or more of cisplatin, carboplatin, and oxaliplatin.

### Compositions

In various embodiments, provided are compositions comprising one or more isolated monomeric platinum (II) or (IV) complexes according to formulas IV, V, VI and VII:

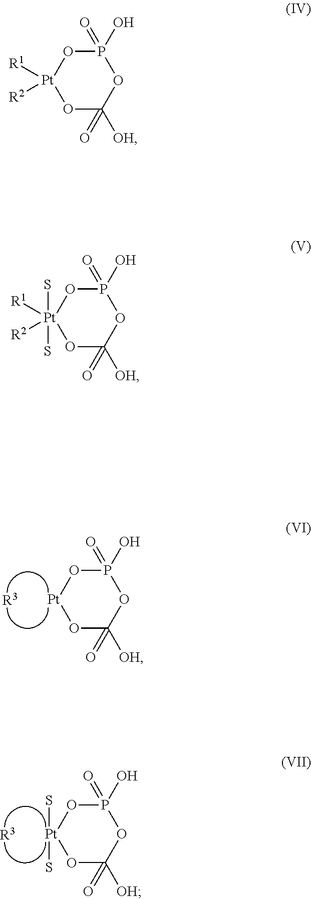

wherein R¹ and R² are each independently selected from NH₃, substituted or unsubstituted aliphatic amines, and substituted or unsubstituted aromatic amines; wherein R¹ and R² are not both NH₃ in formula IV; wherein R³ is selected from substituted or unsubstituted aliphatic or aromatic diamines; and wherein S is independently selected from hydroxide, acetic acid, butyric acid, and alpha-hydroxy acids. Also provided are compositions comprising on or more pharmaceutically acceptable salts or solvates of said complexes.

The phosphaplatins of formulas (VI) and (VII), with platinum coordinated to pyrophosphate and 1,2-cyclohexanediamine ligand, can exist as four stereoisomers due to the possible cis- and trans-geometry of the two amino (—NH₂) groups at the chiral carbon centers 1 and 2 of the diamine ligand. These stereoisomers exhibit the (1R,2R)-, (1S,2S)-, (1R,2S)-, and (1S,2R)-configurations. The trans-ligand (trans-1,2-cyclohexanediamine) affords two enantiomers, having the (1R,2R)- and (1S,2S)-configurations, respectively. The cis-isomer in principle encompasses the (1R,2S)- and (1S,2R)-enantiomers, but these two cis-isomers are equivalent, superimposable mirror images indistinguishable from each other structurally and chemically. Thus, the two enantiomers of the cis-isomer will be referred to hereinafter simply as the “cis-isomer” and are referred to with a single formula.

In some of the various embodiments, the provided compositions have one or more enantiomers of pyrodach-4, but one of skill in the art will recognize that the application is not limited thereto as other phosphaplatins (such as pyrodach-2) may have similar utility. Accordingly, the provided compositions may comprise:

(a) enantiopure (1R,2R)-pyrodach-4 having formula (I);

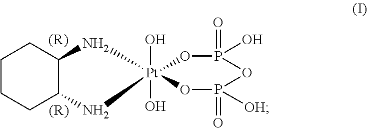

(b) enantiopure (1S,2S)-pyrodach-4 having formula (II);

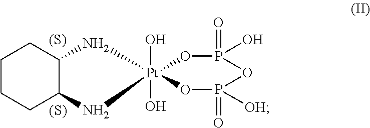

(c) a racemic mixture of pyrodach-4 having equal amounts of (1R,2R)-pyrodach-4 having formula (I) and (1S,2S)-pyrodach-4 having formula (II);

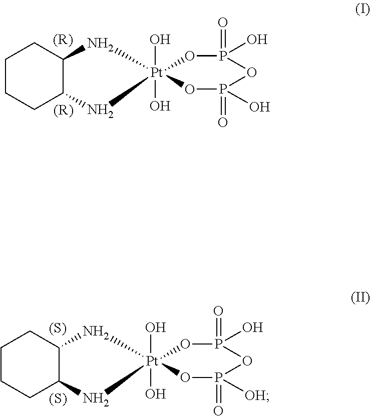

(d) enantioenriched pyrodach-4 having an enantiomeric excess of either (1R,2R)-pyrodach-4 having formula (I) or (1S,2S)-pyrodach-4 having formula (II);

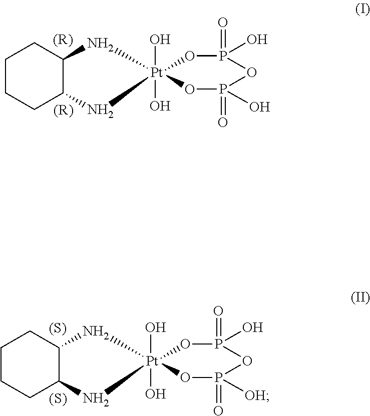

(e) cis-pyrodach-4 having formula (III)

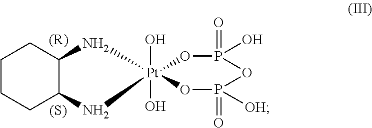

(f) pharmaceutically acceptable salts of any of (a)-(e);
(g) pharmaceutically acceptable solvates of any of (a)-(e); or
(h) combinations thereof.

Referring to the amino groups on the 1,2-cyclohexanediamine ligand in the complexes of formulas (I) and (II), the (1R,2R) and (1S,2S) stereochemistries represent amino groups in trans configurations, whereas in the complexes of formula (III), (1R,2S) and (1S,2R) stereochemistries represent amino groups in cis configurations.

An enantioenriched pyrodach-4 mixture may be characterized as having an enantiomeric excess greater than zero of either the (1R,2R)-enantiomer or the (1S,2S)-enantiomer. The enantiomeric excess may vary and in example embodiments may be greater than 0.01%, greater than 1%, greater than 10%, greater than 25%, greater than 50%, greater than 75%, greater than 90%, greater than 98%, greater than 99%, greater than 99.9%, or even equal to 100%. In example embodiments, the enantiomeric excess may be of the (1R,2R)-enantiomer. In further example embodiments, the enantiomeric excess may be of the (1S,2S)-enantiomer.

As a non-limiting example, the compounds of formulas (IV)-(VII) may be synthesized from a starting material, such as cis-(1,2-cyclohexanediamine) dichloroplatinum(II), which may be prepared by converting K₂PtCl₄ to K₂PtI₄ by the addition of potassium iodide. The K₂PtI₄ may then be reacted with a 1,2-cyclohexanediamine having a desired stereochemistry, such as cis-1,2-cyclohexanediamine, trans-(1R,2R)-1,2-cyclohexanediamine, trans-(1S,2S)-1,2-cyclohexanediamine, or mixtures thereof. The resulting (1,2-cyclohexanediamine)diiodoplatinum(II) complexes then may be transformed to the corresponding (1,2-cyclohexanediamine)diaquaplatinum(II) complexes in situ by adding two equivalents of silver nitrate. The diaqua species [Pt(1,2-cyclohexanediamine)(H₂O)₂] then may be converted to the cis-dichloro [Pt(1,2-cyclohexanediamine)Cl₂] complexes by addition of potassium chloride.

It will be understood that other suitable reaction conditions may be used. In non-limiting examples, the starting 1,2-cyclohexanediamine-platinum(II) complexes were reacted with excess pyrophosphate and the temperature may be from about 35° C. to about 45° C., or any preferred narrower range between 35° C. and 45° C. Good results have been obtained at 40° C. In some examples, the reaction may be allowed to proceed from about 13 hours to about 16 hours, or any preferred narrower range between 13 hours and 16 hours. Good results have been obtained at reaction times of 15 hours. In some examples, the pH can be from about 6 to about 7, from about 7 to about 8, and from about 8 to about 9. Good results have been obtained at pH of about 8.

The aqueous reaction mixture may be concentrated such that precipitates of pyrophosphate do not form. It will be understood that the aqueous reaction mixture may be concentrated in any suitable manner. For example, the aqueous reaction mixture may be concentrated by rotary evaporation.

Thereupon, the pH of the reaction mixture may be lowered rapidly to a pH of less than 2 by addition of a suitable acid. In some examples, nitric acid may be used to lower the pH. In some embodiments, the pH is in the range between about 1 to about 2. Good results have been obtained at pH of 1.

In some examples, the reaction mixture may be cooled to a temperature of between 5° C. and room temperature (25° C.±2° C.) after concentrating the reaction mixture. In other examples, the method also includes cooling the reaction mixture to a temperature of between 5° C. and room temperature after lowering the pH of the reaction mixture.

To prepare the platinum (IV) complexes according to formulas (V and VII, as well as additional steps are required to attach the hydroxo ligands. Thus, in addition to the steps described above, to the reaction mixture may be added hydrogen peroxide, and optionally a reagent selected from the group acetate salts, butyrate salts, and salts of alpha-hydroxy acids, after maintaining the reaction mixture at a temperature of about 30° C. to about 60° C. for a period of about 12 hours to about 18 hours at a pH from about 7 to about 9. The optional reagent that may be added together with hydrogen peroxide prior to concentration of the reaction mixture may be selected from sodium acetate, sodium butyrate, amines, and sodium salts of alpha-hydroxy acids. In other examples, the optional reagent added together with hydrogen peroxide prior to concentration of the reaction mixture may be selected from potassium acetate, potassium butyrate, any monodentate amines such as ammonia, isopropyl amine, and others, and potassium salts of alpha-hydroxy acids.

In some of the various embodiments, the provided compositions may additionally comprise at least one pharmaceutically acceptable ingredient selected from carriers, diluents, adjuvants, and vehicles, which generally refer to inert, non-toxic, solid or liquid fillers, diluents, or encapsulating materials unreactive with the phosphaplatins. These types of additives are well known in the art and are further described below with regard to treatment methods.

In some of the various embodiments, the provided compositions may additionally comprise one or more of cisplatin, carboplatin, and oxaliplatin.

As described elsewhere herein, racemic mixtures of complexes of formulas (I) and (II) have shown promise for being anti-angiogenic agents due to their ability to suppress gene expression of Vascular Endothelial Growth Factor Receptor 2 (VEGFR-2), which is known to play important roles in angiogenesis. Racemic mixtures of complexes of formulas (I) and (II) have also shown promise for being anti-metastatic agents due to their ability to increase E-cadherin, the loss of expression of which is known to be correlated with tumor malignancy in several types of cancers. Additionally, racemic mixtures of complexes of formulas (I) and (II) have shown promise as being pro-apoptotic agents due to their ability to stimulate expression of pro-apoptotic genes PUMA, PTEN, Fas, FasL, and Bax, as well as trigger at least two possible pro-apoptotic signaling pathways. The ability of such complexes to have said effects has also been shown in cancer cells, including those resistant to cisplatin. In light of such advancement of the study and utility of phosphaplatins, as well as similarities between pyrodach-4 and related phosphaplatins [including, but not limited to, enantiopure complexes of formulas (I) and (II), enantioenriched mixtures of complexes of formulas (I) and (II), and complexes of formula (III)], it is contemplated that other phosphaplatins may be, alone or in compositions, useful for inhibiting angiogenesis, inhibiting metastasis, promoting apoptosis, or combinations thereof.

Accordingly, in some of the various embodiments, the provided compositions may be anti-angiogenesis agents for use in preventing or inhibiting angiogenesis, anti-metastatic agents for use in preventing or inhibiting metastasis, or both. In some embodiments, the provided compositions may be useful for targeting development of new vessels or regrowth or restructuring of existing vessels. In some of the various embodiments, the provided compositions may be pro-apoptotic agents for use in promoting apoptosis.

### Manufacture of Medicaments

In some of the various embodiments, the provided compositions may be useful for inhibiting angiogenesis, inhibiting metastasis, or both. The compositions may also be useful for promoting apoptosis. In light of the aforementioned, in some embodiments also provided are uses of (a) enantiopure (1R,2R)-pyrodach-4 having formula (I);

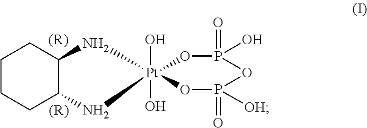

(b) enantiopure (1S,2S)-pyrodach-4 having formula (II);

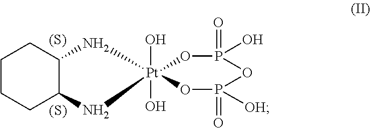

(c) a racemic mixture of pyrodach-4 having equal amounts of (1R,2R)-pyrodach-4 having formula (I) and (1S,2S)-pyrodach-4 having formula (II);

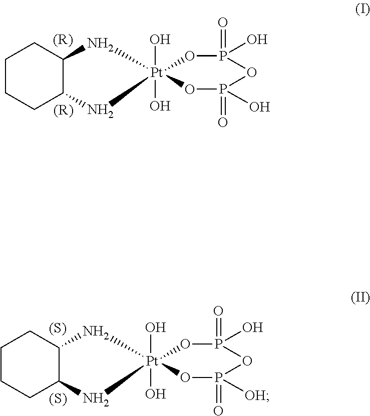

(d) enantioenriched pyrodach-4 having an enantiomeric excess of either (1R,2R)-pyrodach-4 having formula (I) or (1S,2S)-pyrodach-4 having formula (II);

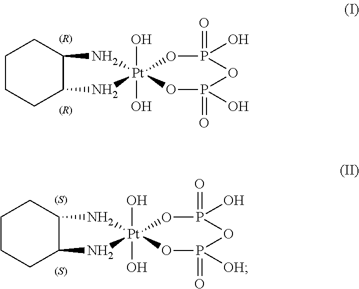

(e) cis-pyrodach-4 having formula (III)

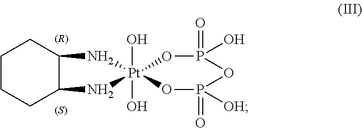

(f) pharmaceutically acceptable salts of any of (a)-(e);
(g) pharmaceutically acceptable solvates of any of (a)-(e); or
(h) combinations thereof;
in the manufacture of a medicament for inhibiting angiogenesis; in the manufacture of a medicament for inhibiting metastasis; in the manufacture of a medicament for inhibiting angiogenesis and metastasis; or in the manufacture of a medicament for promoting apoptosis. In some embodiments, said medicaments may be useful for treating cancers, including those that are resistant to one or more of cisplatin, carboplatin, and oxaliplatin.

### Methods

In still further embodiments, the complexes, compositions, or both, described above may be used alone, or with other pharmaceutically acceptable ingredients, in methods of treatment. The provided methods comprise administering to a subject in need thereof a therapeutically effective amount of a complex or composition described above. The subject may be an animal such as, for example, a mammal, including a human. It is contemplated that such methods may be useful in inhibiting or preventing angiogenesis in endothelial cells of a subject, cancer cells of a subject, or both. Accordingly, such methods may be useful in treating cancers, such as ovarian, brain, stomach, bladder, breast, lung, or pancreatic cancers. In some embodiments, it is contemplated that the complexes and/or compositions may be used in combination therapies involving concurrent or sequential treatment with known platinum-metal drugs such as cisplatin, carboplatin, and/or oxaliplatin. It is further contemplated that the complexes and/or compositions may be used to treat cancers resistant to treatment by one or more of cisplatin, carboplatin, oxaliplatin and/or used in combination with other treatment classes or targeted therapies. In even more specific embodiments, compositions utilized herein with methods described herein can include isolated, monomeric pyrodach-4. In specific embodiments, the therapeutically effective amount or amounts of any chemicals or compositions herein described are administered at a level sufficient to also increase gene expression, in at least one cancer cell of the subject, of at least one of p53 upregulated modulator of apoptosis (PUMA); phosphatase and tensin-homolog (PTEN); tumor necrosis factor receptor superfamily member 6 (FAS), Fas ligand (FasL); caspase-3 (CASP3); caspase-9 (CASP9); and BCL-2-associated X protein (Bax). In even more specific embodiments, the chemicals and compositions herein described can be applied individually or in combination to increase E-cadherin in at least one cancer cell selected from ovarian, brain, stomach, bladder, breast lung, and pancreatic cells.

The provided compositions may be administered and dosed in accordance with good medical practice, taking into account the clinical condition of the individual patient, the site and method of administration, scheduling of administration, patient age, sex, body weight and other factors known to medical practitioners. Administration of the treatment can be performed in a hospital or other medical facility by medical personnel. The pharmaceutically “therapeutic effective amount” for purposes herein is thus determined by such considerations as are known in the art. The amount must be effective to achieve improvement including, but not limited to, improved survival rate or more rapid recovery, or improvement or elimination of symptoms and other indicators as are selected as appropriate measures by those skilled in the art.

It is contemplated that the provided compositions may be administered to animals, including mammals and humans alone or as combinatorial therapies. Moreover, it is contemplated that the compositions may be administered over an especially wide therapeutic window. Of course, one of skill in the art will appreciate that therapeutic dosages may vary by complex administered, composition administered, and subject receiving the administered complex or composition. The doses can be single doses or multiple doses over a period of several days. As an illustrative example, it is contemplated that the compositions may be administered in one, two, three, four, five, six, or more doses in one more days. It is also contemplated that the complexes may be administered continuously over one or more days, such as by a pump or drip. As another illustrative example, it is contemplated that the compositions may be administered for one, two, three, four, five, six, seven, eight, nine, ten, or more days.

In a method of use, it is contemplated that the provided composition can be administered in various ways. It should be noted that phosphaplatins can be administered alone in aqueous solution taking advantage of the excellent solubility of these complexes, or as an active ingredient in combination with pharmaceutically acceptable carriers, diluents, adjuvants and vehicles. It is also contemplated that the compositions can be administered orally, subcutaneously or parenterally including intravenous, intraarterial, intramuscular, intraperitoneally, intratonsillar, and intranasal administration as well as intrathecal and infusion techniques. Implants of the compositions may also be useful.

When the compositions are administered parenterally, they generally will be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for the compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. However, any vehicle, diluent, or additive used would have to be compatible with the phosphaplatin complexes.

Sterile injectable solutions can be prepared by incorporating the phosphaplatin complexes in the required amount of the appropriate solvent with one or more of the other ingredients, as desired.

### EXAMPLES

The described embodiments will be better understood by reference to the following examples, which are offered by way of illustration and which one skilled in the art will recognize are not meant to be limiting.

### I. Evidence for Anti-angiogenesis

### Example 1

Certain phosphoplatins show promise in treatment of angiogenesis such as those related to tumors. Tumor expansion and proliferation invariably depend on angiogenesis. To prevent angiogenesis, a variety of strategies and targets have been adopted in the development of anticancer agents. Among these are the Vascular Endothelial Growth Factors (VEGF) and their Receptors (VEGFR), since these play important roles in angiogenesis. Many human cancers have overexpressed VEGF and its receptor VEGFR-2. VEGFR-2, also known as Flkl and KDR, is a glycoprotein and contains seven immunoglobulin-like extracellular domains and one intracellular tyrosine kinase domain.

In this application we show that VEGRF-2 gene expression is suppressed as much as 50% by pyrodach-4 in both cisplatin-sensitive and -resistant human ovarian cancer cells (Table 1). Since this receptor is primarily expressed in endothelial cells, we also present data from Human Umbilical Vein Endothelial Cells (HUVEC) cells to support the suppression of the VEGFR-2 gene, which indicates that phosphaplatins may be capable of exhibiting antiangiogenesis in all types of cancers. Universally, HUVECs serve as a model for the study of angiogenesis. The data are shown in Table 1 and experimentation methods are discussed in Example 2 below.

TABLE 1
Normalized expression of VEGFR2 gene in cisplatin-sensitive
(A2780), and - resistant (A2780/C30) human ovarian cancers,
and in HUVEC cells. The positive numbers indicate manifold
increase while negative numbers signify relative decrease
in expression compared to untreated cells.

| Conditions | A2780 | A2780/C30 | HUVEC |
|---|---|---|---|
| Control | 1.0 | 1.0 | 1.0 |
| Cisplatin-treatment | 2.5 | 2.0 | — |
| Pyrodach-4-treatment | −2.0 | −2.0 | −2.0 |

### Example 2

Evaluations of selected gene expressions in Human Umbilical Vein Endothelial Cells (HUVEC) and Human Ovarian Cancer Cells by quantitative reverse transcriptase polymerase chain reaction (PCR)

Quantitative real-time PCR experiments were performed to estimate the expression of a selection of targeted genes. HUVEC cells (Lonza Walkersville, Frederick, Md.) were cultured on 0.1% gelatin-coated petri dishes by following standard protocols recommended by the vendor in a humidified incubator by maintaining 5% CO₂ at 37° C. Human epithelial ovarian cancer cells (A2780) and cisplatin-resistant cells (A2780/C30) were cultured in RPMI 1640 with or without cisplatin (7 μM) and pyrodach-4 (180 μM) for 0, 3, 12 and 24 hours. In addition, the two ovarian cancer cell lines were also treated with pyrodach-4 in the presence of PFT-α (15 μM) for 24 hours to study the effect of inhibition of the p53 protein's transcriptional function. HUVECs were treated with or without cisplatin (7 μM) or pyrodach-4 (90 or 180 μM) for various durations ranging from 1 hr. to 6 hrs.

Both treated and untreated cancer cells were maintained in RPMI 1640 medium with 10% fetal bovine serum, 2 μmol L-glutamine, 100 units/mL penicillin-streptomycin and 0.25 units/mL insulin solution, at 37° C. with 5% CO₂. RNA was isolated from both the treated and untreated cells. Treated cells are those cells harvested after the exposure to a phosphaplatin at its IC-50 value concentration at different time intervals, from three hours to 24 hrs. The RNA samples were treated with DNase-free RNase (QIAGEN Sciences) to remove DNA. cDNA was then synthesized by using the High Capacity RNA-to-cDNA kit (Applied Biosystems) according to the manufacturer's instructions. The quantity and purity of RNA were determined by UV spectroscopy (NanoDrop 2000 Thermo Scientific, Wilmington, Del.). A minimum absorbance ratio index (ratio of the absorbance measured at 260 nm over that at 280 nm) of 1.9 was used as an acceptable purity. The isolated RNA samples were stored at −80° C. and were subject to minimal freeze-thaw cycles to maintain RNA integrity. Duplex real-time PCRs were performed using Taqman® Gene Expression Assays for the target gene and endogenous control (β-actin) in the same reaction well. The endogenous control is the reference used to normalize the target mRNA. These chain reactions were initially performed using different cDNA concentrations to determine the optimal concentrations of cDNA required to detect the gene of interest. The target gene was labeled with a blue dye (FAM, absorbance: 494 nm, emission: 518 nm) while the reference gene was tagged with a green dye (VIC, absorbance: 538 nm, emission: 554 nm). The cDNA concentrations were selected such that the threshold cycle (Ct) values for the target genes were between 17 and 32, most sensitive for the fluorescence detection by ABI StepOnePlus™ instrument used for the measurements.

The threshold cycle number (Ct), the point at which the fluorescence of the qPCR reaction just exceeds the threshold fluorescence of the detection system, was determined. These Ct values were used to compare the levels of target genes and the endogenous controls. Quantitative gene expressions are reported in terms of fold-change (2^{-ΔΔCt}) which were calculated from ΔCt and ΔΔCt values using: ΔCt=(Ct target−Ct reference) and ΔΔCt=(ΔCt)time X−(ΔCt)time zero (control). The fold-expression for the control samples (untreated) remained uniform since the value of ΔΔCt=0, and therefore 2°=1.

For the HUVEC cell treated with pyrodach-4, notable observations include suppression of VEGFR-2 expression by as much as 50%, and over-expression of PUMA by 10-15 fold; PTEN, 4-8 fold; Fas, 2-5 fold; FasL, 2-4 fold; caspase-3 and -9 2-3 fold, and Bax 2-3 fold. The levels of other genes stated above remained unchanged within the experimental errors. These additional gene expressions have been discussed, in part, in previous literature on our compounds, and are further discussed below.

### II. Evidence for Activation of Tumor Suppressor PTEN Gene by Phosphaplatins

### Example 3

### Experimental Methods are Discussed in Example 2 Above

Phosphatase and tensin homolog (PTEN) plays an important role in tumorigenesis. The PTEN gene is located on chromosome ten and encodes a cytoplasmic enzyme with both protein and lipid phosphatase activity. The gene is frequently mutated or deleted in many malignant cancers including gastric and glioblastomas. In fact, the mutation and deletion of PTEN accounts for as much as 80% of human glioblastomas. Introduction of the PTEN gene in glioblastoma has shown to inhibit the vascularization even in the presence of proangiogenic stimuli.

PTEN is a lipid phosphatase that dephosphorylates phosphatidylinositol-3,4,5-trisphosphate (PIP3) to phosphatidylinositol-4,5-bisphosphate (PIP2) and therefore negatively controls the anti-apoptotic phosphatidylinositol 3-kinase(PI3K)/Akt pathways. In recent years, tremendous efforts have been directed to inhibit PI3K pathways for treating cancers, with several drugs in different phases of clinical trials targeting the PI3K kinase. Such a strategy, however, must be carefully executed since severe deregulation of PI3K activities can be linked with a variety of severe side effects, including triggering diabetes, schizophrenia, Parkinson's disease, and many other diseases. As stated earlier, PTEN works both by antagonizing PI3K and negatively regulating Akt. Therefore, the activation of PTEN, which would silence Akt (downstream of PI3K), may be more strategic in treating cancer without creating many of the side effects stated above. This is because the PI3K can still provide its vital functions, in this way, since the kinase can be activated by at least three independent pathways (although all of them require receptor tyrosine kinases (RTKs)).

We provide evidence of significant overexpression of PTEN in both sensitive and resistant human ovarian cancers mediated by phosphaplatins. Furthermore, this overexpression of PTEN is controlled by p53, since PTF-α, an inhibitor of p53, abolished the overexpression. When A2780 and A2780/C30 cells were treated with pyrodach-4, the expression of the PTEN gene was elevated to 5.5 and 2.3 fold compared to control.

TABLE 2
Normalized expression of PTEN in human ovarian cancers,
in the presence of treatment by Phosphaplatins,
and in the presence of a p53 inhibitor.

| Condition | A2780 | A2780/C30 |
|---|---|---|
| Control | 1.0 | 1.0 |
| Pyrodach-4-treatment | 5.5 | 2.3 |
| Pyrodach-4 + PTF-α | 0.8 | — |

### III. Evidence for Anti-Metastatic Properties of Phosphaplatins

### Example 4

There is a strong correlation between the loss of expression or function of E-cadherin (also known as Arc-1, cell-CAM 80/120, and uvomorulin) and tumor malignancy in several different types of cancers including bladder, breast, lung and pancreatic cancers. E-cadherin primarily functions as a cell-cell adhesion molecule and prevents dedifferentiation and invasiveness of carcinomas. In contrast, N-cadherin (a mesenchymal cadherin) exhibits opposite effects: it promotes cell mobility, migration, and invasion. Although there is still much to be learned about how E-cadherin loses its expression and function, the concept of a ‘cadherin switch’ from epithelial to mesenchymal cadherins has been proposed to explain the transition of a benign to an invasive and malignant tumor. The invasiveness, in fact, can be reversed by forced expression of E-cadherin in tumor cell lines that have lost the E-cadherin function or expression.

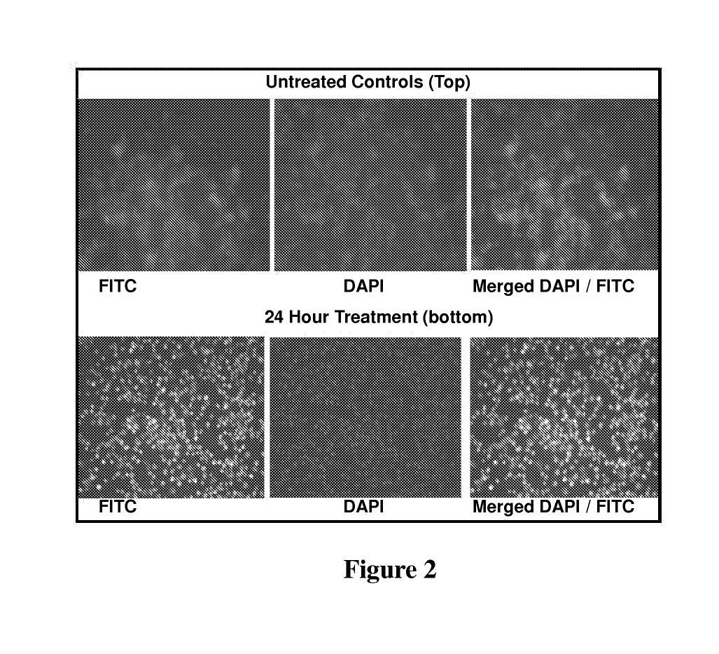

In this invention, we present immunofluorescent data to indicate that pyrodach-4 not only increases the expression but also reduces the diffusivity of E-cadherin in human ovarian cancer cell. FIG. 2 shows the comparative immunofluorescent images of E-cadherin protein before and after treatment of A2780 cells with pydodach-4 for 24 hr. Left panels show E-cadherin bound to its antibody labeled with FITC (green). The middle and right panels represent nuclei stained with Prolong® Gold Antifade Reagent with 4′-6-Diamidino-2-phenylindole (DAPI, a nuclear stain) and merger of the left and middle panels (see Example 5 below for details).

### Example 5

### Direct and Indirect Immunofluorescent Labeling of proteins

Cells were grown on multi-chamber slide systems (Lab-Tek™, Nunc, Denmark). These cells were treated with pyrodach-4 at or below IC50 concentrations for 24 hours. Untreated cells were used as a control while treated and untreated cells without primary antibody labeling were used as negative controls. Cells were fixed with 4% formaldehyde in PBS for 10 minutes at room temperature, washed with PBS (Ca2+ and Mg2+ free) and blocked in blocking buffer (5% FBS) for 2 hours at room temperature. Subsequently, incubation with the primary antibodies, diluted in antibody dilution buffer (1% BSA) at 1:1000 dilution, was done for an hour at room temperature. The Fas and E-cadherin proteins were detected via direct labeling using anti-Fas (FITC labeled, Santa Cruz Biotechnology, Inc., Santa Cruz, Calif.) and anti-E-cadherin (FITC labeled, BD Transduction Laboratories, Franklin Lakes, N.J.) fluorescently tagged antibodies. Other proteins, p53, BAX and PUMA were detected indirectly with fluorescently labeled secondary antibodies (Texas Red-conjugated anti-mouse IgG for p53 and Fluorescein-conjugated anti-rabbit IgG for BAX and PUMA, obtained from Vector Labs, Burlingame, Calif.) at 1:1000 dilution. Following the second incubation for an hour with the secondary antibodies, the fixed cells were carefully washed once with PBS, the nuclei were stained with Prolong® Gold Antifade Reagent with DAPI (Invitrogen, Carlsbad, Calif.). The stain was cured for 24 hours before fluorescence images were acquired. Images of immuno-stained cells were obtained at 400-fold magnification, using a Nikon microscope, SPOT camera and SPOT Advanced imaging software (Westchester, Ohio).

### IV. Evidence of Anti-resistant Properties of Phosphaplatins

### Example 6

Although platinum-based chemotherapeutics are widely used, inherent and acquired resistance to platinum metal drugs limit their application. For example, a significant percentage of patients becomes resistant to cisplatin treatment. The current literature supports the hypothesis that the ability to repair DNA damage by excising bound platinum from DNA mostly contributes to the resistance mechanisms. This hypothesis is supported by a large body of evidence that cisplatin treated cancer cells or cancerous tissues taken from patients show increased sensitivity among the nucleotide excision repair genes (NER), homologous recombination repair (HRR), and post-replication repair (PRR). These genes include: NER (RAD2, RAD4, RAD10, RAD1, and RAD14), HRR(RAD57, RAD55, RAD51, RAD52, RAD54, and RAD59), and PRR(RAD6, RAD18, and RAD5).

In this invention, we show that none of the DNA damage genes showed detectable sensitivity in response to phosphaplatins treatment. This conclusion is based on genome-wide fitness assays in yeast in the presence of cisplatin, pyrodach-2 and pyrodach-4 to evaluate the sensitivity of the NER, HRR, and PRR genes. Cisplatin indeed induced expression of those genes related to the DNA response. The profiles of both Phosphaplatin compounds were unique in that not a single gene involved in DNA repair or other DNA metabolic processes showed any sensitivity as a mutant. For both compounds the sensitive strains were significantly enriched for the following functions; 1) iron and copper transport and 2) metal-regulated transcription factors. In addition, pyrodach-2 induced sensitivity in the strain avo2, a component of the Tor kinase complex. Overall, these profiles showed few sensitive strains, suggesting that even at this high dose, they are well tolerated by the cell. The detailed experiment is illustrated in Example 7.

### Example 7

### Genome-Wide Fitness Assay Description

The around 6000 strains in the yeast deletion collection can be studied in a single culture by using a microarray to detect the 20 bp DNA “barcodes” or “tags” contained in each strain. Barcode intensities are compared across time-points or across conditions to analyze the relative fitness of each strain. This development of the pooled fitness assay has greatly facilitated the functional annotation of the yeast genome by making genome-wide gene-deletion studies faster and easier, and has led to the development of high throughput methods for studying drug action in yeast. Pooled screens can be used for identifying gene functions, measuring the functional relatedness of gene pairs in order to group genes into pathways, identifying drug targets, and determining a drug's mechanism of action. This process involves five main steps: preparing aliquots of pooled cells, pooled growth, isolation of genomic DNA and PCR amplification of the barcodes, array hybridization (or next generation sequencing), and data analysis.

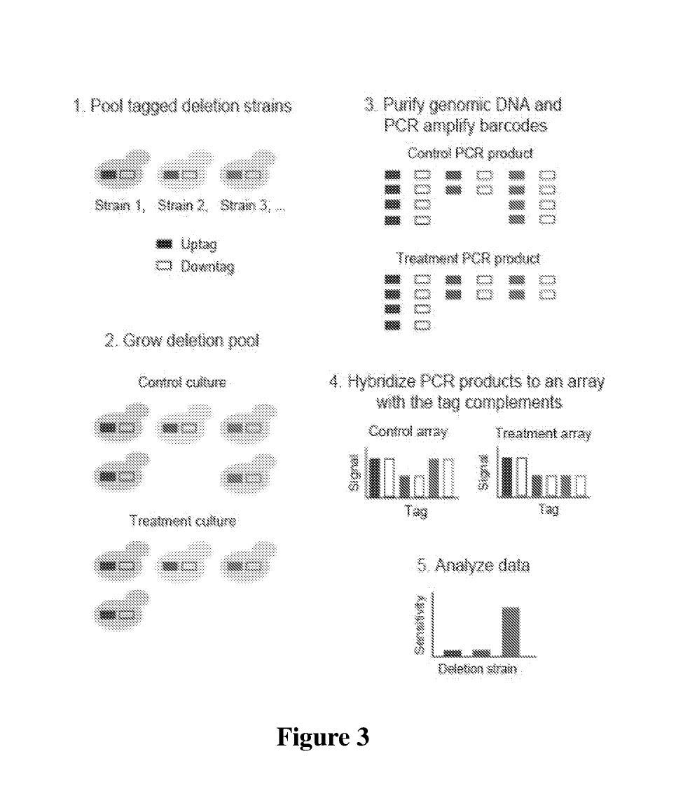

The pooled fitness assay involves five main steps (FIG. 3). First, the deletion collection is grown on solid media in arrayed format and the resulting cells are pooled and frozen to make cell aliquots that will be used for starting growth experiments. Cells are then grown in the desired conditions, typically for 5 to 20 generations. The barcodes from the resulting cell samples are prepared for hybridization by isolation of the genomic DNA and PCR amplification of the barcodes. The barcodes are amplified in two reactions to prevent crosstalk between the uptag and downtag primers. The resulting PCR products are then hybridized to tag arrays with one array needed per cell sample. Data is then analyzed to determine differences in strain representation between pairs of samples.

To analyze the genome wide effects of pyrodach-2 and -4, we screened 2 collections of strains, 1200 essential genes as heterozygote deletion mutants (reducing functional gene dose from 2 to 1) and 4800 non-essential genes as complete deletions (reducing functional gene dose from 2 to 0). Both compounds were screened at the highest dose possible (200 μM final concentration) to increase the likelihood of finding compound-specific sensitive strains.

FIG. 3 shows an overview of the pooled fitness assay. Fitness profiling of pooled deletion strains involves six main steps:
1. Strains are first pooled at approximately equal abundance.
2. The pool is grown competitively in the condition of choice and a control condition. If a gene is sensitive to the treatment condition, the strain carrying this deletion will grow more slowly and become under-represented relative to the control culture (purple strain). Resistant strains will grow faster and become over-represented (not shown).
3. Genomic DNA is isolated from cells harvested at the end of pooled growth, and barcodes are amplified from the genomic DNA with universal primers.
4. PCR products are then hybridized to an array that detects the tag sequences, giving tag intensities for the two samples.
5. The treatment and control sample are then compared to determine the relative fitness of each strain. Note that only the purple strain is described as sensitive to the condition. While the red strain grows more slowly than the blue strain in the treatment, this growth difference in not of interest because it matches that seen in the control.

### V. Evidence for Two Parallel Pro-Apoptotic Signaling Pathways Induced by Phosphaplatins in their Anti-Cancer Activity

### Example 8

A single anti-tumor agent capable of inducing two or more signaling pathways is a better approach to treat cancers for a variety of reasons. First, many drugs develop resistance to treatment by activating specific genes in response to therapeutics. If an agent activates multiple signaling pathways, development of resistance to multiple genes that are not involved in crosstalk would be less likely. Also, complete inhibition of key enzymes that have multiple functions might lead to severe side effects as usually observed for many anticancer drugs.

In this invention, we present data that confirm that phosphaplatins trigger at least two possible pro-apoptotic pathways, in addition to rendering anti-angiogenic and anti-metastatic properties. First, our compounds showed significant overexpression of pro-apoptotic genes such as Fas, P53, Bax, PUMA, and PTEN. To test whether all these pro-apoptotic genes belong to a single signaling pathway, gene expressions were followed in the presence of PFT-α, an inhibitor of p53. Table 3 shows the expression of some key pro-apoptotic genes in the presence and absence of this p53 inhibitor. As can be seen from the Table 3, in the presence of the inhibitor of p53, Fas maintained significant overexpression, albeit less than in its absence. However, the inhibitor significantly suppresses the expression of PUMA, PTEN and Bax. These results indicate that Fas is functioning by invoking both p53-dependent and p53-independent pathways. Furthermore, PUMA is also functioning in both a p53-dependent and p53-independent manner. Therefore, even after shutting down the p53 and/or PTEN-pathways, pyrodach-4 is still capable of initiating apoptosis. This conclusion is further verified by the ELISA assay in the presence of a p53 inhibitor.

TABLE 3
Expression of key genes modulated by pyrodach-4 in the presence and
absence of p53 inhibitor, PTF-α. The positive numbers represent
manifold increase while negative numbers signify decrease in expression
compared to the expressions of genes in untreated cells.

| | Pyrodach-4 | Pyrodach-4 + 15FM |
|---|---|---|
| Test Genes | treatment only | PFT-α |
| BAX | 2.8 | 1.0 |
| Bcl-2 | 1.0 | −2.0 |
| BID | 1.8 | 1.0 |
| Caspase 3 | 2.6 | −1.2 |
| Caspase 9 | 2.0 | 1.0 |
| Fas | 11.0 | 5.5 |
| FasL | 4.6 | undetectable |
| p53 | 1.8 | 2.1 |
| PUMA | 16.6 | 4.4 |
| VEGFR-2 | −1.9 | −1.6 |
| PTEN | 5.5 | −1.3 |

### Example 9

### Verification of Independent Signaling Mechanisms for Apoptosis by Cell Death Detection Assay with ELISA

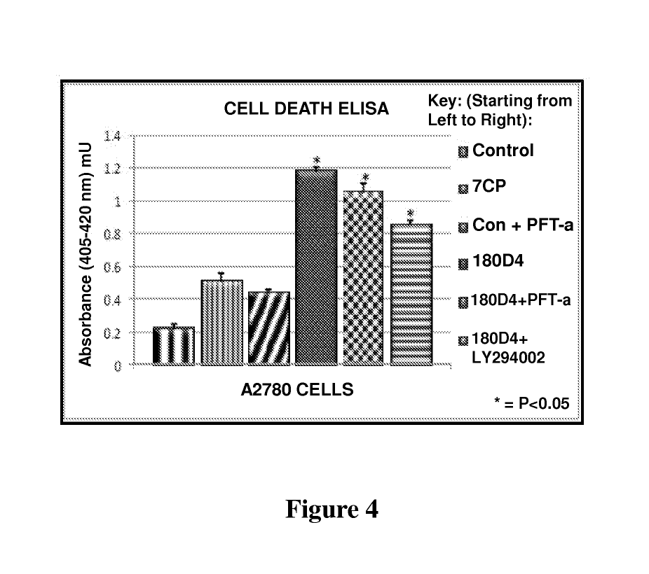

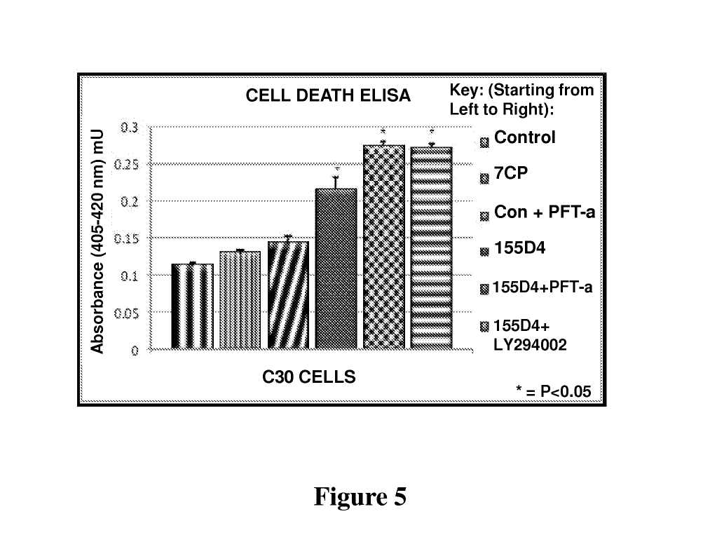

To support the gene expression data, which suggest that the p53 inhibitor does not completely arrest the apoptotic effect of phosphaplatins, cell death experiments with ELISA were performed on both A2780 and A2780/C30 cancer cells. In these experiments, ovarian cancer cells were treated with pyrodach-4 in the presence and absence of inhibitors PFT-α (an inhibitor p53) and LY29400 (an inhibitor of PI3K). The data are shown in FIGS. 4 and 5.

FIG. 4 shows an ELISA assay of apoptotic cell death in A2780 cells induced by pyrodach-4 and cisplatin in the absence and presence of p-53 (PFT-α, 15 μM) and PI3K (LY294002, 30 μM) inhibitors. Cells were treated with compounds for 24 hr. The different treatment groups from left to right are: control, 7 μM cisplatin, Con+PFT-α, 180 μM D4, 180 μM D4+PFT-α, 180 μM D4+LY294002. Statistical analysis was conducted using the Tukey test. N=3 and P<0.05. In the figure, “*” indicates values significant from untreated control.

FIG. 5 shows an ELISA assay of apoptotic cell death in A2780/C30 cells induced by pyrodach-4 and cisplatin in the absence and presence of p-53 (PFT-α, 15 μM) and PI3K (LY294002, 30 μM) inhibitors. The different treatment groups from left to right are: control, 7 μM cisplatin, Con+PFT-α, 180 μM D4, 180 μM D4+PFT-α, 180 μM D4+LY294002. Apoptosis was quantified using the Cell Death Detection ELISA from Roche. Statistical analysis was conducted using the Tukey test. N=3 and P<0.05. In the figure, “*” indicates values significantly from untreated control.

The y-axis of FIGS. 4 and 5 represents the absorbance which is proportional to cell death. As can be seen, in the presence of p53 and LY-294002, there was some reduction in cell death for A2780 but the overall cell death remained extremely high compared to cisplatin. Note that A2780/C30 cells are truly refractory to cisplatin treatment, with an insignificant increase in absorbance observed when cells were treated with 7 FM cisplatin, while pydodach-4 unambiguously showed cell death. It is also intriguing to note that the inhibitors have no effects on the resistant ovarian cells.

These results indicate that Phosphaplatins function in multiple pathways in killing cancer cells and therefore internal mechanisms which might shut down a specific path may only have minimum effect on treatment of cancers with the compounds of the present invention. The details are elaborated in Example 10.

### Example 10

### Cell Death Detection Assay with ELISA

A2780 and A2780/C30 cells were grown in monolayers, trypsinized and suspended in supplemented RPMI 1640 media as described in Example 5. All the treatments on the Fc/A2780 and Fc/A2780/C30 cell samples were carried out in triplicate. The ELISA was performed according to the manufacturer's instructions. Briefly, 50,000 cells were plated for each treatment and the cells were allowed to adhere for 4-6 hours. Cells were then either incubated with media alone (untreated sample) or with pyrodach-4 or cisplatin in the concentrations indicated above. DNA of the treated and untreated cells was assayed for nucleosomal DNA using the ELISA kit (purchased from Roche Diagnostics, Mannheim, Germany). The cells were lysed for 30 minutes using the lysis buffer. After centrifuging, 10% (v/v) supernanant was incubated with anti-histone-biotin and anti-DNA-peroxidase. After two hour incubation, the pellets were washed three times and incubated with the substrate solution. The absorbances were measured at 405 and 490 nm for each treatment type using SPECTRA Max M2 multichannel fluorescence plate reader (Molecular Devices, Sunnyvale, Calif.). The absorbances of the treated samples were normalized to the substrate solution. One way ANOVA with Tukey multiple comparison tests were performed using Minitab 16. P values≦0.05 were deemed significant.

This application should not be considered limited to the specific examples described herein, but rather should be understood to cover all aspects of the invention. Various modifications, equivalent processes, as well as numerous structures and devices to which the present invention may be applicable will be readily apparent to those of skill in the art. Those skilled in the art will understand that various changes may be made without departing from the scope of the invention, which is not to be considered limited to what is described in the specification.

1-2. (canceled)

3. The method of claim 30, comprising:
administering to a subject in need of inhibition of angiogenesis a therapeutically effective amount of a composition comprising:
(a) (1R,2R)-pyrodach-4 having formula (I) 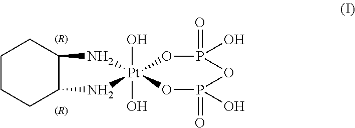
or a pharmaceutically acceptable salt or solvate thereof;
(b) (1S,2S)-pyrodach-4 having formula (II) 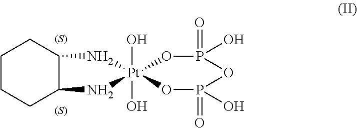
or a pharmaceutically acceptable salt or solvate thereof; or
(c) combinations thereof;
wherein the therapeutically effective amount administered is sufficient to modify gene expression, in at least one cancer cell of the subject, of at least one surface molecule associated with angiogenesis.

4. The method of claim 3, wherein the at least one cancer cell is selected from ovarian, brain, stomach, bladder, breast, lung, and pancreatic cancer cells.

5. The method of claim 3, wherein the at least one cancer cell is resistant to at least one of cisplatin, carboplatin, and oxaliplatin.

6. The method of claim 3, wherein the therapeutically effective amount administered is sufficient to also increase gene expression, in the at least one cancer cell of the subject, of at least one of p53 upregulated modulator of apoptosis (PUMA); phosphatase and tensin-homolog (PTEN); tumor necrosis factor receptor superfamily member 6 (Fas), Fas ligand (FasL); caspase-3 (CASP3); caspase-9 (CASP9); and BCL-2-associated X protein (Bax).

7. The method of claim 4, wherein the at least one cancer cell is resistant to at least one of cisplatin, carboplatin, and oxaliplatin.

8. The method of claim 3, wherein the therapeutically effective amount administered is sufficient to decrease gene expression of Vascular Endothelial Growth Factor Receptor-2 (VEGFR-2) surface molecule.

9. The method of claim 3, wherein said composition further comprises at least one pharmaceutically acceptable ingredient selected from carriers, diluents, adjuvants, and vehicles.

10. The method of claim 3, wherein said composition further comprises cisplatin, carboplatin, oxaliplatin, or combinations thereof.

11-12. (canceled)

13. The method of claim 3, wherein said composition comprises isolated, monomeric pyrodach-4.

14. The method of claim 30, comprising:
administering to a subject in need of inhibition of metastasis a therapeutically effective amount of a composition comprising:
(a) (1R,2R)-pyrodach-4 having formula (I) 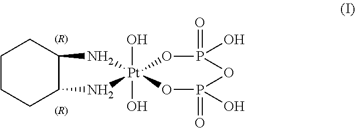
or a pharmaceutically acceptable salt or solvate thereof;
(b) (1S,2S)-pyrodach-4 having formula (II) 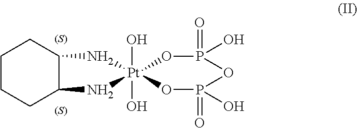
or a pharmaceutically acceptable salt or solvate thereof;
(c) combinations thereof;
wherein the therapeutically effective amount administered is sufficient to increase E-cadherin in at least one cancer cell of the subject.

15. The method of claim 14, wherein E-cadherin is increased in at least one cancer cell selected from ovarian, brain, stomach, bladder, breast, lung, and pancreatic cancer cells.

16. The method of claim 15, wherein the at least one cancer cell is resistant to at least one of cisplatin, carboplatin, and oxaliplatin.

17. The method of claim 14, wherein the therapeutically effective amount administered is sufficient to also increase gene expression, in the at least one cancer cell of the subject, of at least one of p53 upregulated modulator of apoptosis (PUMA); phosphatase and tensin-homolog (PTEN); tumor necrosis factor receptor superfamily member 6 (Fas), Fas ligand (FasL); caspase-3 (CASP3); caspase-9 (CASP9); and BCL-2-associated X protein (Bax).

18. The method of claim 14, wherein said composition further comprises at least one pharmaceutically acceptable ingredient selected from carriers, diluents, adjuvants, and vehicles.

19. The method of claim 14, wherein said composition further comprises cisplatin, carboplatin, oxaliplatin, or combinations thereof.

20-21. (canceled)

22. The method of claim 14, wherein said composition comprises isolated, monomeric pyrodach-4.

23. A method of inhibiting angiogenesis and metastasis, comprising:
administering to a subject in need thereof a therapeutically effective amount of a composition comprising:
(a) (1R,2R)-pyrodach-4 having formula (I) 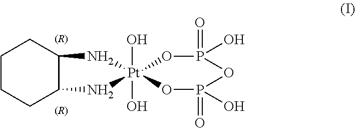
or a pharmaceutically acceptable salt or solvate thereof;
(b) (1S,2S)-pyrodach-4 having formula (II) 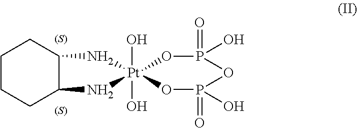
or a pharmaceutically acceptable salt or solvate thereof;
(c) cis-pyrodach-4 having formula (III) 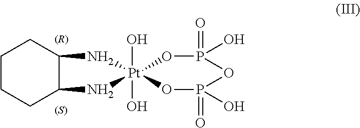
or a pharmaceutically acceptable salt or solvate thereof; or
(d) combinations thereof;
wherein the therapeutically effective amount administered is sufficient to decrease gene expression of Vascular Endothelial Growth Factor Receptor-2 (VEGFR-2) and increase E-cadherin in at least one cancer cell of the subject.

24. The method of claim 23, wherein the at least one cancer cell is selected from ovarian, brain, stomach, bladder, breast, lung, and pancreatic cancer cells.

25. The method of claim 24, wherein the at least one cancer cell is resistant to at least one of cisplatin, carboplatin, and oxaliplatin.

26. The method of claim 23, wherein said composition further comprises at least one pharmaceutically acceptable ingredient selected from carriers, diluents, adjuvants, and vehicles.

27. The method of claim 23, wherein said composition further comprises cisplatin, carboplatin, oxaliplatin, or combinations thereof.

28-29. (canceled)

30. A method of inhibiting angiogenesis or metastasis, comprising contacting cells of a tissue or sample from a subject with a compound selected from:
(a) (1R,2R)-pyrodach-4 having formula (I) 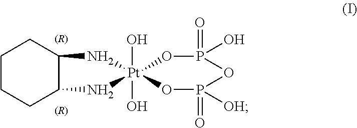
or a pharmaceutically acceptable salt or solvate thereof;
(b) (1S,2S)-pyrodach-4 having formula (II) 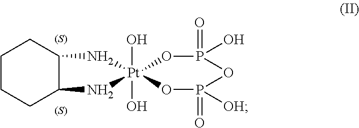
or a pharmaceutically acceptable salt or solvate thereof; and
(c) cis-pyrodach-4 having formula (III) 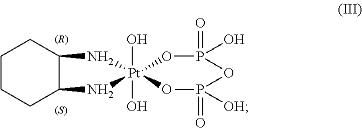
or a pharmaceutically acceptable salt or solvate thereof.

31. The method of claim 30, wherein said tissue or sample is from a tumor.

32-33. (canceled)

34. A method of inducing apoptotic cell death in proliferative cells, comprising contacting the proliferative cells with a compound selected from:
(a) (1R,2R)-pyrodach-4 having formula (I) 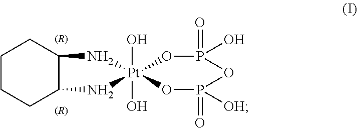
or a pharmaceutically acceptable salt or solvate thereof;
(b) (1S,2S)-pyrodach-4 having formula (II) 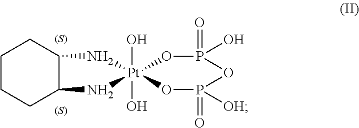
or a pharmaceutically acceptable salt or solvate thereof; and
(c) cis-pyrodach-4 having formula (III) 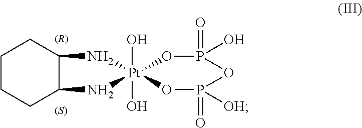
or a pharmaceutically acceptable salt or solvate thereof.

35. The method of claim 34, wherein said proliferative cells are obtained from or in a subject having a proliferative disease.

36-37. (canceled)

38. The method of claim 34, wherein said proliferative cells are selected from ovarian, brain, stomach, bladder, breast, lung, and pancreatic cancer cells.
